(19) United States
(12) Patent Application Publication
(10) Pub. No.: US 2022/0211162 A1
(43) Pub. Date: Jul. 7, 2022

(54) SKIN CARE APPARATUS

(71) Applicant: LG HOUSEHOLD & HEALTH CARE LTD., Seoul (KR)

(72) Inventors: Eun Mi KIM, Seoul (KR); Kyung Sik JANG, Seoul (KR); Bok Hyun PACK, Seoul (KR)

(73) Assignee: LG HOUSEHOLD & HEALTH CARE LTD., Seoul (KR)

(21) Appl. No.: 17/607,684

(22) PCT Filed: Apr. 22, 2020
(86) PCT No.: PCT/KR2020/005274

§ 371 Date: Oct. 29, 2021

(30) Foreign Application Priority Data
May 16, 2019 (KR) 10-2019-0057276

(51) Int. Cl. A45D 44/00; A61H 23/02

(52) U.S. Cl. CPC A45D 44/002; A61H 23/02; A61H 2205/022; A45D 2200/155; A61H 2201/0207; A45D 2200/207

(57) ABSTRACT

The present invention is for providing a skin care apparatus which provides skin care focusing on eye rims, forehead and the like. The skin care apparatus comprises: a main body; and a mounting body for mounting the main body on a user's face. A sheet accommodation part for accommodating a sheet is formed on the main body. The main body comprises at least one first area coming into contact with a sheet accommodated in the sheet accommodation part and a second area between the edge of the main body and the first area. The thickness of the first area can be smaller than the thickness of the second area.

### TECHNICAL FIELD

The present invention relates to a skin care apparatus, and in particular, to a skin care apparatus capable of intensive care of eye rims and forehead.

### BACKGROUND ART

With the development of beauty industry, people's interest is increasing in skin care services for improving skin tone or wrinkles as well as products such as basic cosmetics and color cosmetics.

Meanwhile, the effect of skin care generally appears when the skin care is received continuously for a long period of time rather than when the skin care is received for a short period of time. Therefore, it is important to receive continuous skin care in order for a user to acquire effects such as skin tone improvement or wrinkle improvement.

However, users have difficulty in continuing to receive skin care due to reasons such as lack of access to places where skin care services are provided and lack of time.

Therefore, it is required to develop a skin care apparatus that provides a skin care function at a place desired by the user and at a time desired by the user.

### DISCLOSURE

### Technical Problem

An object of the present invention is to provide a skin care apparatus that is easy to use.

An object of the present invention is to provide a skin care apparatus that cares for the skin such as eye rims, forehead, and the like.

An object of the present invention is to provide a skin care apparatus capable of customized care for each skin area such as eye rims, forehead, and the like.

An object of the present invention is to provide a skin care apparatus that may be used by adhering a sheet to a face.

An object of the present invention is to provide a skin care apparatus capable of not only bringing a sheet into contact with a face, but also giving a heating effect or a vibration effect to a portion of the face with which the sheet is in contact.

### Technical Solution

A skin care apparatus according to an embodiment of the present invention includes a main body, and a mounting body for mounting the main body on a user's face, wherein the main body is formed with a sheet accommodation part in which a sheet is accommodated, the main body includes at least one first area with which the sheet accommodated in the sheet accommodation part is in contact, and a second area between an edge of the main body and the first area, and a thickness of the first area may be smaller than a thickness of the second area.

The thickness of the first area may be an average thickness of the first area, and the thickness of the second area may be an average thickness of the second area.

The main body may be formed with an opening corresponding to the user's eyes, and at least a part of the first area may overlap the opening in a vertical direction.

The first area is provided to the main body in pairs, the pair of first areas are spaced apart from each other in left and right directions, and the main body includes a left area including the first area positioned on the left of the pair of first areas, a right area including the first area positioned on the right of the pair of first areas, and a central area between the left area and the right area, wherein an average thickness of the central area may be greater than each of an average thickness of the left area and an average thickness of the right area.

The main body may further include an outer body, an inner body disposed on a rear surface of the outer body and having an inner surface facing the user's face, and a vibration module disposed between the outer body and the inner body.

The main body may further include an outer body, an inner body disposed on a rear surface of the outer body and having an inner surface facing the user's face, and a heat generating module disposed between the outer body and the inner body.

A thickness of the inner body may be smaller than a thickness of the outer body.

The heat generating module may overlap the first area in a horizontal direction.

The sheet accommodation part may further include a rib for guiding the mounting of the sheet.

A thickness of the rib may be smaller than a thickness of the sheet.

The sheet accommodation part may be formed with a recessed portion for accommodating the sheet.

A depth of the recessed portion may be smaller than the thickness of the sheet.

The sheet may be detached/attached from/to the sheet accommodation part.

The main body may be formed of silicone, thermo plastic elastomer (TPE), or urethane.

A skin care apparatus system according to an embodiment of the present invention includes a terminal, and a skin care apparatus including a communication module for transmitting and receiving a signal to and from the terminal, wherein the skin care apparatus includes a main body, and a mounting body for mounting the main body on a user's face, and the main body is formed with a sheet accommodation part in which a sheet is accommodated, the main body includes at least one first area with which the sheet accommodated in the sheet accommodation part is in contact, and a second area between an edge of the main body and the first area, and a thickness of the first area may be smaller than a thickness of the second area.

The skin care apparatus may further include a vibration module mounted on the main body, and a processor for setting an operation method of the vibration module based on a control command received from the terminal.

The processor may set vibration intensity of the vibration module disposed at a position corresponding to the first area to be different from vibration intensity of the vibration module disposed at a position corresponding to the second area.

The skin care apparatus may further include a heat generating module mounted on the main body, and a processor for setting an operation method of the heat generating module based on a control command received from the terminal.

The skin care apparatus may further include a processor for setting an operation method based on at least one piece of environmental information and user information received from the terminal.

### Advantageous Effects

According to the present invention, by forming a thickness of an area with which a sheet is in contact to be smaller than a thickness of an area with which the sheet is not in contact, the area with which the sheet is not in contact prevents a skin care apparatus from being excessively pressed against a face of a user, thereby serving as a frame. In addition, in this case, since a separate additional member for the frame is not required, there is advantage that an assembly process can be simplified and manufacturing costs can be reduced.

In addition, since the thickness of the area with which the sheet is in contact is formed to be smaller, vibration or heat generated from a vibration module or a heat generating module disposed around the sheet can be more easily transmitted to the sheet, and accordingly, there is advantage that it is possible to improve a rate at which ingredients contained in the sheet are absorbed by the skin.

In addition, since thicknesses of both side areas of a main body are formed to be smaller than a thickness of a central area thereof, the bending of both side areas can be easier than that of the central area, and accordingly, there is an advantage that it is possible to adhere the entire main body to the face regardless of different face shapes and face sizes for each user.

It is possible to minimize loss in a process of transferring vibration or heat generated from the vibration module or the heat generating module to the user's face by forming a thickness of an inner body to be smaller than a thickness of an outer body, and accordingly, there is an advantage that it is possible to protect the vibration module or the heat generating module from the outside.

A rib or a recessed portion can guide an attachment position when the user attaches the sheet to a sheet accommodation part, and accordingly, there is an advantage that it is possible to provide convenience. In addition, there is an advantage that it is possible to maximize a contact area between the sheet and the user's face by forming a thickness of the rib or a depth of the recessed portion to be smaller than a thickness of the sheet.

### DESCRIPTION OF DRAWINGS

FIG. 1 is a perspective view illustrating a front of a skin care apparatus according to an embodiment of the present invention.

FIG. 2 is a perspective view illustrating a rear surface of the skin care apparatus according to the embodiment of the present invention.

FIG. 3 is a perspective view illustrating a main body according to an embodiment of the present invention.

FIG. 4 is a perspective view illustrating a state in which the main body and a sheet shown in FIG. 3 are attached.

FIG. 5 is a cross-sectional view taken along line A-A′ of FIG. 3.

FIG. 6 is a cross-sectional view taken along line B-B′ of FIG. 3.

FIG. 7 is a cross-sectional view taken along line C-C′ of FIG. 3.

FIG. 8 is a perspective view illustrating a main body according to another embodiment of the present invention.

FIG. 9 is a cross-sectional view taken along line D-D′ of FIG. 8.

FIG. 10 is a view illustrating a main body and a mounting body in which an inner body and an outer body are separated according to the embodiment of the present invention.

FIG. 11 is a control block diagram of a skin care apparatus according to an embodiment of the present invention.

FIG. 12 is a view illustrating a piezoelectric sensor that may be provided in the skin care apparatus according to the embodiment of the present invention.

FIG. 13 is a view illustrating an inner surface of a main body according to another embodiment of the present invention.

FIG. 14 is a view illustrating a contact time sensing device that may be provided in a skin care apparatus according to an embodiment of the present invention.

FIG. 15 is a view illustrating an optical vibration composite device that may be provided in the skin care apparatus according to the embodiment of the present invention.

FIG. 16 is a view illustrating a tunable light emitting module that may be provided in the skin care apparatus according to the embodiment of the present invention.

FIG. 17 is a view illustrating a skin thickness measurement device that may be provided in the skin care apparatus according to the embodiment of the present invention.

FIGS. 18A to 18B are views illustrating a linear ultrasound module that may be provided in the skin care apparatus according to the embodiment of the present invention.

FIG. 19 is a view illustrating a composite skin thickness measurement device that may be provided in the skin care apparatus according to the embodiment of the present invention.

FIG. 20 is a control block diagram of a skin care apparatus system including the skin care apparatus according to the embodiment of the present invention.

### MODES OF THE INVENTION

Hereinafter, the present invention will be described in more detail with reference to the drawings.

The suffixes “module” and “part” for the elements used in the following description are given in consideration of only the ease of creating the specification, and do not impart a particularly important meaning or role by themselves. Therefore, the “module” and “part” may be used interchangeably with each other.

The terms “first,” “second,” etc. may be used herein to describe various elements, but these elements should not be limited by these terms. These terms are only used to distinguish one element from another.

A singular expression includes a plural expression unless the context clearly refers to otherwise.

It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

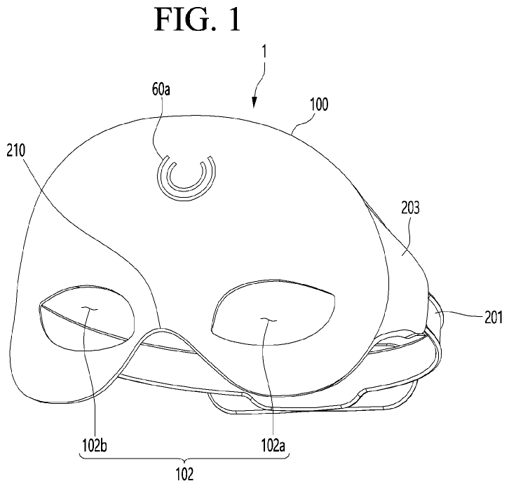

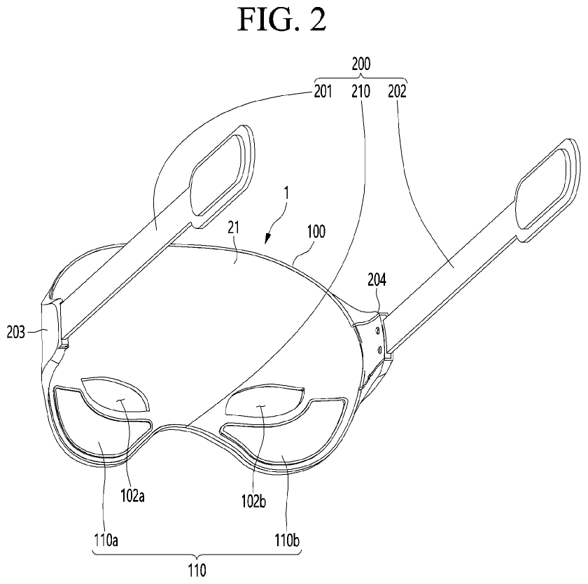

FIG. 1 is a perspective view illustrating a front of a skin care apparatus according to an embodiment of the present invention. FIG. 2 is a perspective view illustrating a rear surface of the skin care apparatus according to the embodiment of the present invention.

A skin care apparatus 1 according to the embodiment of the present invention may include a main body 100 and a mounting body 200.

At least a portion of the main body 100 may be in contact with a user's face and provide a skin care function to the user's face. A stimulation providing unit for providing the skin care function to the user's face may be disposed on the main body 100. The stimulation providing unit provides stimulation to the user's face and the skin of the user. The stimulation providing unit may include a module that generates vibration, light, heat, and the like.

In addition, the main body 100 may be provided with a power button 60a, and a power module 60 may be turned on or off according to an input of pressing the power button 60a. As the power module 60 is turned on, various components provided in the skin care apparatus 1 may be supplied with power. A shape and position of the power button 60a are merely illustrative, and thus the embodiment is not limited thereto.

An opening 102 corresponding to the user's eyes may be formed in the main body 100. The opening 102 may include a left opening 102a corresponding to the user's left eye and a right opening 102b corresponding to the user's right eye.

The opening 102 may secure a field of view of the user mounting the skin care apparatus 1.

The mounting body 200 may be connected to the main body 100. Meanwhile, according to the embodiment, the skin care apparatus 1 may further include connection bodies 203 and 204 for connecting the main body 100 and the mounting body 200.

The mounting body 200 may mount the main body 100 on the user's face.

According to an embodiment, the mounting body 200 may be connected to both side ends of the main body 100 and may be mounted to surround the head of the user. In this case, the mounting body 200 may be made of a material such as rubber with elasticity and may be formed in a band shape. Therefore, when the skin care apparatus 1 is mounted on the user, the mounting body 200 may closely contact the main body 100 toward the user's face.

The mounting body 200 may include a left mounting member 201 and a right mounting member 202. The left mounting member 201 may be closely connected to the left opening 102a of the left opening 102a and the right opening 102b, and the right mounting member 202 may be closely connected to the right opening 102b of the left opening 102a and the right opening 102b.

When the skin care apparatus 1 further includes the connecting bodies 203 and 204, the connecting bodies 203 and 204 may include a left connecting body 203 connecting the left mounting member 201 to the main body 100 and a right connecting body 204 connecting the right mounting member 202 to the main body 100.

Meanwhile, the left connection body 203 and the right connection body 204 may be integrally formed with the main body 100.

Meanwhile, the mounting body 200 may further include a nose mounting portion 210. The nose mounting portion 210 may be mounted and supported on the user's nose, and in this case, the main body 100 may be mounted on the user's face.

The nose mounting portion 210 may be formed in a shape in which a lower end of the main body 100 is recessed between the left opening 102a and the right opening 102b. When the skin care apparatus 1 is mounted on the user's face, the user's nose may be positioned at a recessed portion between the left opening 102a and the right opening 102b.

Meanwhile, the main body 100 may be made of a flexible material. For example, the main body 100 may be formed of a material such as silicone, thermo plastic elastomer (TPE), or urethane, but it is merely illustrative for convenience of description, and the main body 100 may be formed of various materials capable of being adhered to the user's face. When the main body 100 is formed of a material such as silicone, thermo plastic elastomer (TPE), or urethane, there is advantage that it may be waterproof so that washing with water is easy.

Similarly, the mounting body 200 may also be made of a flexible material. As described in the main body 100, the mounting body 200 may be formed of a material such as silicone, TPE, or urethane, but it is merely illustrative for convenience of description, and thus the embodiment is not limited thereto.

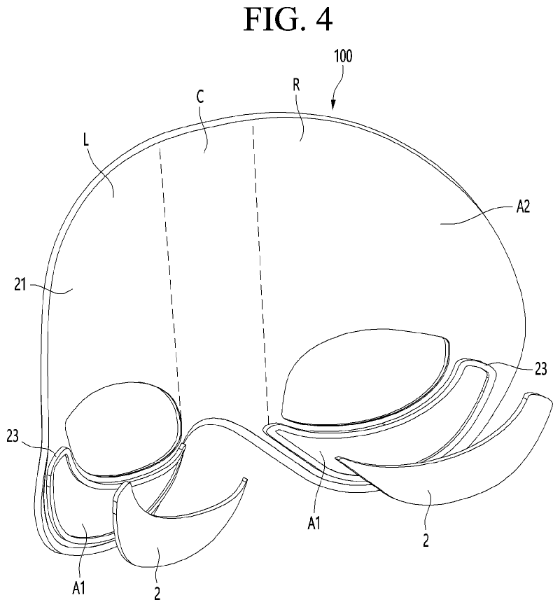

The main body 100 may be formed with a sheet accommodation part 110 in which a sheet 2 (see FIG. 4) is accommodated. The sheet 2 may be detached/attached from/to the sheet accommodation part 110.

The sheet accommodation part 110 may be a guide for guiding a position of the sheet 2 when the sheet 2 is attached to the main body 100. The sheet accommodation part 110 may be a sheet fixing guide for preventing the sheet 2 attached to the main body 100 from moving or falling down.

The sheet 2 may be made of a material containing nutrients and moisturizing ingredients for skin care and the material may be made of a nonwoven fabric such as cotton or cloth, or a hydrogel.

According to the embodiment, the sheet 2 may include a microneedle containing an effective ingredient. A material of the microneedle may be a solid-type such as a metal material, silicone, and the like, or may be a soluble-type that is dissolved when applied to the skin using a self-degradable material, a biodegradable material, or the like. A wrinkle improvement and whitening efficacy material such as botox, retinol, hyaluronic acid, and the like may be included in the microneedle. When the microneedle is in close contact with the skin, the efficacy material may penetrate deep into the skin. When the skin care apparatus 1 proposed in the present invention is worn on the face, the microneedles may be smoothly attached and absorbed by making the skin care apparatus 1 to be in close contact with and to press the face.

Meanwhile, the shape of the sheet 2 may be the same as the shape of the sheet accommodation part 110. Specifically, a shape of the outer circumference of the sheet 2 may be the same as a shape of the inner circumference of the sheet accommodation part 110.

As described above, the sheet 2 formed in the same shape as the sheet accommodation part 110 may be referred to as a dedicated product used only for the production of the skin care apparatus 1.

In addition, the sheet 2 formed in the same shape as the sheet accommodation part 110 as described above may be referred to as a dedicated product used only for the use of the skin care apparatus 1.

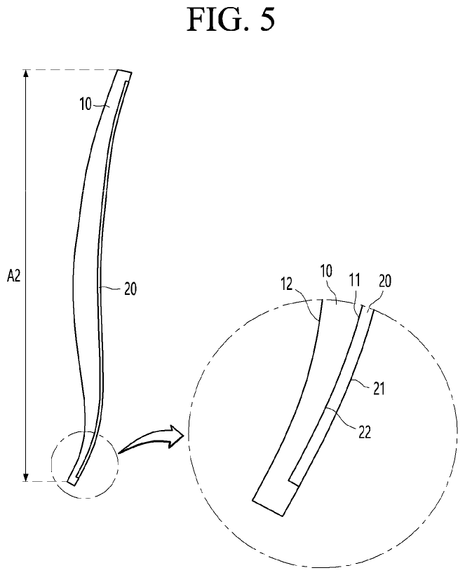

The sheet accommodation part 110 may be formed on a surface that is in contact with the user's face when the main body 100 is mounted on the user's face. The sheet accommodation part 110 may be formed on a rear surface of the main body 100, in particular, on an inner surface 21 of an inner body 20 (see FIG. 5) to be described later.

As shown in FIG. 2, the sheet accommodation part 110 may be formed below the opening 102. The sheet accommodation part 110 may include a left sheet accommodation part 110a formed below the left opening 102a and a right sheet accommodation part 110b formed below the right opening 102b.

However, a position of the sheet accommodation part 110 shown in FIG. 2 is merely illustrative, and the sheet accommodation part 110 may be formed above the opening 102 or formed between the left opening 102a and the right opening 102b.

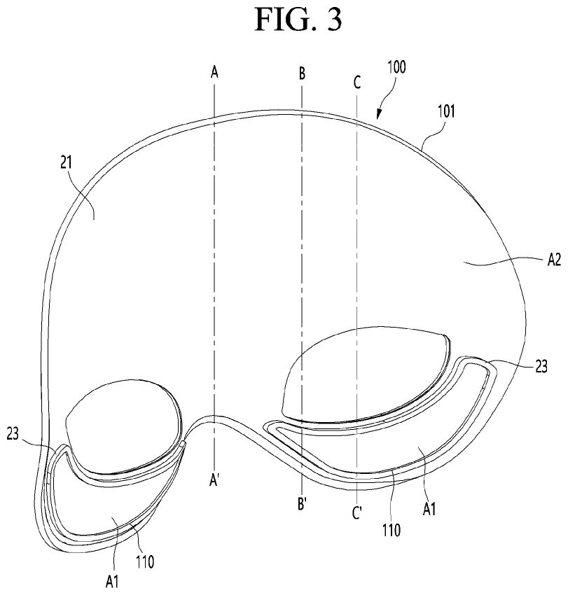

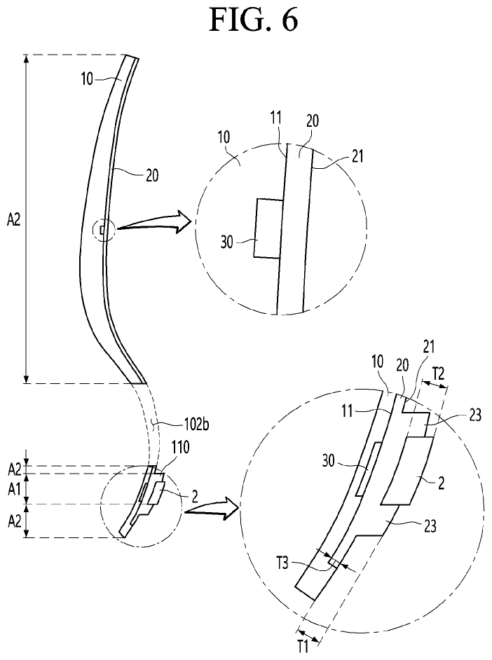

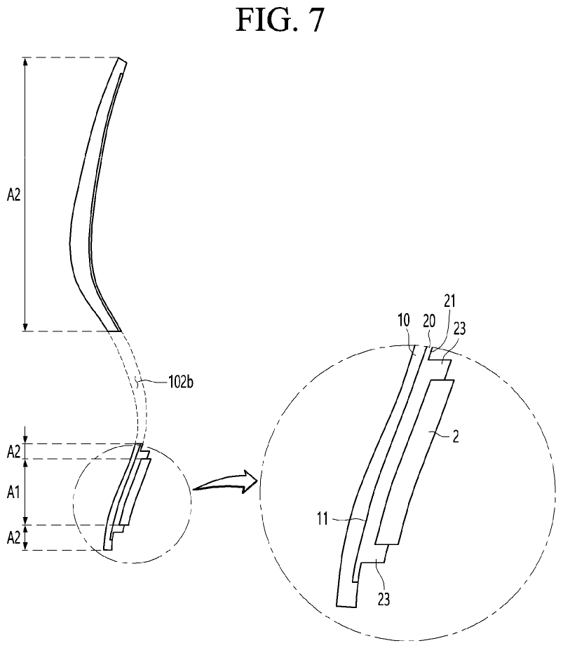

FIG. 3 is a perspective view illustrating a main body according to an embodiment of the present invention, FIG. 4 is a perspective view illustrating a state in which the main body and a sheet shown in FIG. 3 are attached, FIG. 5 is a cross-sectional view taken along line A-A′ of FIG. 3, FIG. 6 is a cross-sectional view taken along line B-B′ of FIG. 3, and FIG. 7 is a cross-sectional view taken along line C-C′ of FIG. 3.

The main body 100 may include at least one first area A1 with which the sheet 2 accommodated in the sheet accommodation part 110 is in contact and a second area A2 with which the sheet 2 is not in contact. The second area A2 may be an area between an edge 101 of the main body 100 and the first area A1.

At least a part of the first area A1 may overlap the opening 102 in a vertical direction.

The first area A1 may be a surface with which the sheet 2 is in contact and a surface that is waterproofed so that ingredients contained in the sheet 2 are not absorbed into the main body 100. Alternatively, a material of the first area A1 and a material of the second area A2 may be different from each other.

According to the embodiment, the sheet accommodation part 110 may include a rib 23 guiding the attachment of the sheet 2. The rib 23 may be formed in the second area A2. The rib 23 may be formed to protrude from the inner surface 21 toward the user's face. The rib 23 may be formed along the outer circumference of the first area A1.

A cross-sectional shape of the first area A1 may be the same as that of the sheet 2. An area of the first area A1 may be greater than or equal to an area of the sheet 2.

The sheet 2 may be attached to at least some or all of the first area A1. In a state in which the sheet 2 is attached to the first area A1, the main body 100 may be mounted on the user's face, and the sheet 2 may be adhered to the user's face by the mounting body 200.

The first area may be provided to the main body in pairs, the pair of first areas may be spaced apart from each other in left and right directions. One of the pair of first areas A1 may be formed in the left sheet accommodation part 110a, and the other of the pair of first areas A1 may be formed in the right sheet accommodation part 110b.

Meanwhile, as shown in FIG. 4, the main body 100 may be divided into three areas. However, the imaginary line dividing the three areas shown in FIG. 4 is merely illustrative, and thus the embodiment is not limited thereto.

When the main body 100 is divided into three areas, the main body 100 may include a left area L including the first area A1 positioned on the left of the pair of first areas A1, a right area R including the first area A1 positioned on the right of the pair of first areas A1, and a central area C between the left area L and the right area R.

In this case, an average thickness of the central area C may be greater than each of an average thickness of the left area L and the average thickness of the right area R. The average thickness of the left area L and the average thickness of the right area R may be the same. When the main body 100 is mounted on the user's face, the main body 100 may be bent according to the shape of the face and may be adhered to the user's face. At this time, since a degree of bending of both areas L and R of the main body 100 is greater than a degree of bending of the central area C, the average thickness of both areas L and R is formed smaller than the average thickness of the center area C, and accordingly, there is an advantage that it is possible to provide wearing convenience.

For example, the maximum thickness of the central area C may be 5.26 mm, and the maximum thickness of both side areas L and R may be 5.18 mm.

Meanwhile, the thickness of the first area A1 may be smaller than that of the second area A2.

In particular, the main body 100 may have the central area of the forehead that is formed to be the thickest. A thickness of the central area of the forehead may be 5T (=5 mm) or more.

Meanwhile, the main body 100 may include an outer body 10 and an inner body 20. The main body 100 may be formed in a form in which the outer body 10 and the inner body 20 are coupled.

The stimulation providing unit may be disposed on the main body 100. The stimulation providing unit may include a vibration module 30 (see FIG. 6), and a heat generating module 40 (see FIG. 7) to be described later. At least one of the vibration module 30 (see FIG. 6) and the heat generating module 40 (see FIG. 7) may be disposed between the outer body 10 and the inner body 20.

The outer body 10 may have a rear surface 11 that is at least partially in contact with the inner body 20 and an outer surface 12 that is opposite to the rear surface 11. When the main body 100 is mounted on the user's face, the outer surface 12 may be the outermost surface.

The inner body 20 may have the inner surface 21 facing the user's face and a mounting surface 22 that is an opposite surface to the inner surface 21. At least a portion of the mounting surface 22 may be in contact with the rear surface 11 of the outer body 10.

The stimulation providing unit such as the vibration module 30 may be disposed on the rear surface 11 of the outer body 10 or the mounting surface 22 of the inner body 20. The stimulation providing unit may include a temperature control module 40, an ion module 42, a far-infrared ray module 44, a light emitting module 46, and the like that are described later in addition to the vibration module 30.

According to an embodiment, the main body 100 may be manufactured in a manner that the outer body 10 covers the inner body 20 after the vibration module 30 is disposed on the mounting surface 22 of the inner body 20. According to another embodiment, the main body 100 may be manufactured in a manner that the inner body 20 is coated on the outer body 10 after the vibration module 30 is disposed on the rear surface 11 of the outer body 10. That is, the vibration module 30 may be disposed at an arbitrary position between the inner body 20 and the outer body 10. In addition, according to the embodiment, the vibration module 30 may also be mounted on the inner surface 21 of the inner body 20.

Referring to FIG. 6, the vibration module 30 may be disposed to protrude from the mounting surface 22 of the inner body 20 toward the outer body 10.

The vibration module 30 may be disposed in at least one of the first area A1 and the second area A2.

For example, eight vibration modules 30 may be installed in the skin care apparatus 1, and first to second vibration modules 30 may be disposed in an area corresponding to the first area A1 positioned on the left, third to fourth vibration modules 30 may be disposed in the area corresponding to the first area A1 positioned on the right, and fifth to eighth vibration modules 30 may be disposed in an area corresponding to the second area A2. The fifth to eighth vibration modules 30 may be disposed to be spaced apart from each other in the left-right direction in the area corresponding to the second area A2 and may be disposed side by side in a row. However, the number and position of the vibration modules 30 are merely illustrative, and thus the embodiment is not limited thereto.

Meanwhile, a thickness T1 of the rib 23 may be smaller or equal to a thickness T2 of the sheet 2.

In particular, as shown in FIG. 6, the thickness T1 of the rib 23 may be smaller than the thickness T2 of the sheet 2, and in this case, the effect that the sheet 2 is adhered to the user's face may be improved. Specifically, when the thickness T1 of the rib 23 is greater than the thickness T2 of the sheet 2, the rib 23 are first in contact with the user's face, and thus a separate space between the sheet 2 and the user's face may be formed by the rib 23. Therefore, the facial adhesion effect of the sheet 2 when the thickness T1 of the rib 23 is smaller than the thickness T2 of the sheet 2 may be higher than the facial adhesion effect of the sheet 2 when the thickness T1 of the rib 23 is greater than the thickness T2 of the sheet 2.

Meanwhile, a thickness T3 of the inner body 20 excluding the rib 23 may be smaller than the thickness T1 of the rib 23 and the thickness T2 of the sheet 2. In this case, there is an advantage that the effect of the stimulation providing unit such as the vibration module 30 disposed between the outer body 10 and the inner body 20 may be effectively transmitted to the user's face. Specifically, since the vibration module 30 is disposed between the outer body 10 and the inner body 20, vibration generated from the vibration module 30 should pass through the inner body 20 to be transmitted to the user's face. Therefore, as the thickness T3 of the inner body 20 is smaller, the effect of the vibration module 30 transmitted to the user's face may be improved.

Meanwhile, the temperature control module 40 may be further disposed on the mounting surface 22 of the inner body 20. For example, the temperature control module 40 may be disposed to protrude from the mounting surface 22 of the inner body 20 toward the outer body 10.

The temperature control module 40 may be disposed at a position corresponding to eye rims, temples, forehead, an area between eyebrows, acupuncture points, etc. As an example, the temperature control module 40 may be disposed at a position overlapping the first area A1 in a horizontal direction. For example, two temperature control modules 40 may be installed in the skin care apparatus 1, and a first temperature control module 40 may be disposed in an area corresponding to the first area A1 positioned on the left. In addition, a second temperature control module 40 may be disposed in an area corresponding to the first area A1 positioned on the right. However, the number and positions of the temperature control modules 40 are merely illustrative, and thus the embodiment is not limited thereto. According to the embodiment, the temperature control module 40 may include a heat generating element, and the heat generating element may discharge heat having a heat generation temperature. In this case, the heat generation temperature may be a temperature between 24° C. and 39° C., which is easily absorbed into the skin and may have a temperature at which moisturizing ingredients may be absorbed into the skin. It is possible to promote absorption of nutrients, moisturizing ingredients, and the like contained in the sheet 2 into the user's face through heat generation.

According to another embodiment, the temperature control module 40 may include an element (e.g., a thermoelectric element) that performs a cooling function. In this case, the temperature control module 40 may cool at least one portion of the user's face.

That is, the temperature of the temperature control module 40 may be set differently depending on a function, a skin condition of the user, and an environmental condition (season, etc.). It is possible to generate effects such as alleviating fatigue around the eyes and wrinkles due to a cool-feeling during cooling and to generate effects such as improvement of blood circulation around the eyes and alleviating dark circles during heating. Meanwhile, the temperature control module 40 may operate together with the vibration module 30, and in this case, there is an advantage that a massage function may also be provided to the user.

Since the thickness T1 of the rib 23, the thickness T2 of the sheet 2, the thickness T3 of the inner body 20 excluding the rib 23, etc. are the same as described in FIG. 6, duplicated descriptions are will be omitted.

In addition, as shown in FIGS. 6 and 7, the thickness of the first area A1 may be smaller than that of the second area A2. The thickness of the first area A1 may be an average thickness of the first area A1, and the thickness of the second area A2 may be an average thickness of the second area A2.

In the case in which the main body 100 is formed of a flexible material, when the main body 100 is in close contact with the user's face, a portion of the nose or forehead that is relatively severely flexed or has protruding bones is excessively pressed, and thus a problem that causes inconvenience to the user may occur.

Therefore, the present invention has an advantage that may minimize excessive pressing of the main body 100 and protect the face by forming the thickness of the second area A2 that is in close contact with the nose, forehead, etc. to be greater than the thickness of the first area A1. In addition, the present invention has an advantage that may improve the effect of the vibration module 30 and the sheet 2 transmitted to the user's face by forming the thickness of the first area A1 to which the sheet 2 is attached to be smaller than the thickness of the second area

A2.

Therefore, since the skin care apparatus 1 according to the embodiment of the present invention does not include a frame, the number of parts may be reduced compared to the case of including the frame inside the main body 100, and in this case, there is an advantage that may simplify the assembly process and reduce a cost.

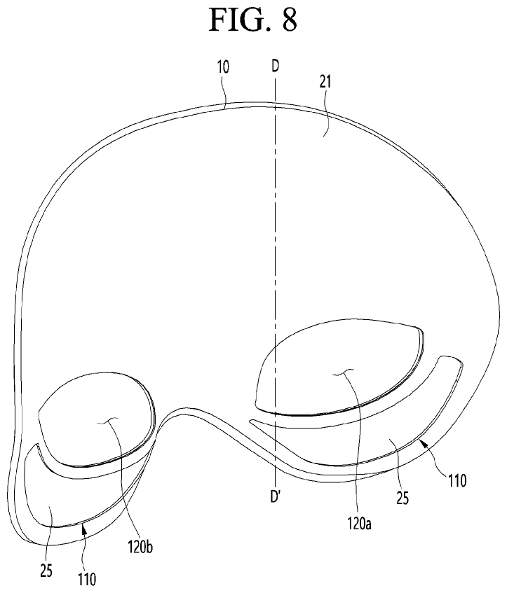

Meanwhile, unlike shown in FIGS. 2 to 7, the sheet accommodation part 110 may not include the rib 23, and a recessed portion 25 (see FIG. 8) for accommodating the sheet 2 may be formed in the sheet accommodation part 110.

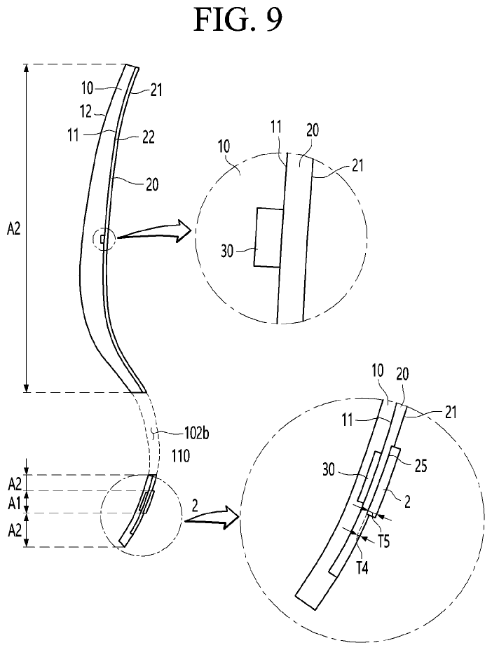

FIG. 8 is a perspective view illustrating a main body according to another embodiment of the present invention, and FIG. 9 is a cross-sectional view taken along line D-D″ of FIG. 8.

According to another embodiment of the present invention, the recessed portion 25 in which the sheet 2 is accommodated may be formed in the sheet accommodation part 110.

The recessed portion 25 may be formed to be recessed from the inner surface 21 of the inner body 20. The sheet 2 may be accommodated in the recessed portion 25 to be attached to the inner body 20. In this case, as described above, the main body 100 may include the first area A1 in contact with the sheet 2 and the second area A2 with which the sheet 2 is not in contact. The second area A2 may be an area between the edge 101 of the main body 100 and the first area A1.

The thickness of the first area A1 with which the sheet 2 accommodated in the sheet accommodation part 110 is in contact may be smaller than the thickness of the second area A2 with which the sheet 2 is not contact. A detailed description of this is the same as described above, and thus a duplicate description will be omitted.

Meanwhile, a depth T4 of the recessed portion 25 may be smaller than the thickness T2 of the sheet 2. In this case, since the sheet 2 protrudes toward the user's face rather than the inner surface 21 when the sheet 2 is accommodated in the recessed portion 25, the effect that the ingredients contained in the sheet 2 is absorbed into the face may be promoted.

FIG. 10 is a view illustrating a main body and a mounting body in which an inner body and an outer body are separated according to the embodiment of the present invention.

An outer openings 102a-1 and 102b-1 may be formed in the outer body 10, and an inner openings 102a-2 and 102b-2 may be formed in the inner body 20.

As shown in FIG. 10, the vibration module 30 may be disposed between the outer body 10 and the inner body 20. For example, the vibration module 30 may be mounted on the mounting surface 22 of the inner body 20.

Meanwhile, the skin care apparatus may further include a circuit board 99, and the circuit board 99 may be disposed on any one connection body 204 as shown in FIG. 10.

The circuit board 99 may be a flexible PCB (FPCB).

However, a shape and position of the circuit board 99 are merely illustrative. According to the embodiment, the circuit board 99 may be disposed on the mounting surface 22 of the inner body 20.

The circuit board 99 may be connected to the power module 60 and the vibration module 30 through at least one of a power supply line (not shown) and a signal line (not shown).

The power module 60 may be connected to the circuit board 99 and the vibration module 30 through the power supply line (not shown) to supply power to the circuit board 99 and the vibration module 30.

The vibration module 30 may be connected to the power module 60 through the power supply line (not shown).

The power module 60 may be disposed on the mounting surface 22 of the inner body 20. The power module 60 may be disposed at a position overlapping the power button 60a provided on the outer body 10 in a horizontal direction.

Meanwhile, a communication module 50 may be further disposed on the connection bodies 203 and 204. However, the communication module 50 may be disposed on the mounting surface 22.

The position of the power module 60 and a position of the communication module 50 shown in FIG. 10 are merely illustrative for convenience of description, and thus the embodiment is not limited thereto.

The power module 60 may include a battery connecting part (not shown) from/to which a battery is detached/attached and may supply power supplied from the battery connected to the battery connecting part to the vibration module 30.

As described above, the power module 60 may supplied with power from the outside in various ways. The above example is merely illustrative for convenience of description, and thus it is reasonable that the embodiment is not limited thereto.

The power module 60 may be turned on/off by the power button 60a. The user may turn on or off power of the skin care apparatus 1 through the power button 60a.

The power module 60 may be controlled so as to operate the vibration module 30 when the power of the skin care apparatus 1 is controlled to be turned on through the power button 60a, and the power module 60 may be controlled so as to stop the operation of the vibration module 30 when the power of the skin care apparatus 1 is controlled to be turned off through the power button 60a.

The vibration module 30 may vibrate for a set time. The vibration module 30 may be in direct/indirect contact with the user's skin to transmit vibration. The vibration module 30 may induce vibration by a vibrator that vibrates by an electric signal.

The skin care apparatus 1 may include a plurality of vibration modules 30, and when the skin care apparatus 1 includes the plurality of vibration modules 30, each of the plurality of vibration modules 30 may set individually the vibration intensity and the number of vibrations. Through this, there is an advantage that customized care for each skin region is possible by adjusting at least one of the vibration intensity and the number of vibrations according to the characteristics of the skin on which the vibration module 30 is disposed.

For example, it is assumed that the skin care apparatus 1 includes eight vibration modules 30, four vibration modules 30 are disposed in the first area A1, and the remaining four vibration modules 30 are disposed in the second area A2. According to an embodiment, the vibration intensity and number of vibrations of the vibration module 30 disposed in the first area A1 may be set to be the same as the vibration intensity and number of vibrations of the vibration module 30 disposed in the second area A2. According to another embodiment, the vibration intensity of the vibration module 30 disposed in the first area A1 may be set to be smaller than the vibration intensity of the vibration module 30 disposed in the second area A2. According to still another embodiment, the number of vibrations of the vibration module 30 disposed in the first area A1 may be set to be greater than the number of vibrations of the vibration module 30 disposed in the second area A2. However, the above-described examples are merely illustrative for convenience of description, and thus the embodiment is not limited thereto.

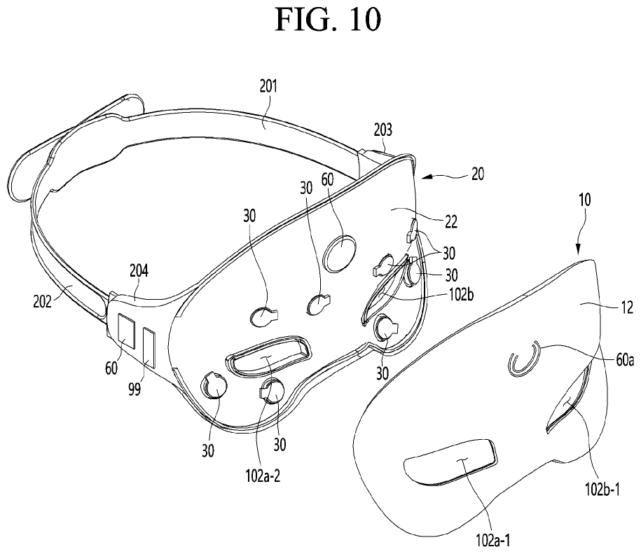

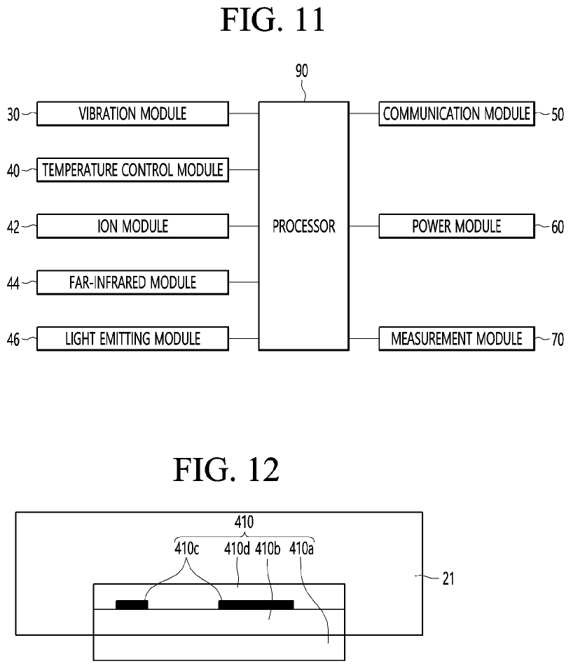

In addition, the skin care apparatus 1 may further include a stimulation providing unit other than the vibration module 30. This will be described in detail in FIG. 11.

FIG. 11 is a control block diagram of a skin care apparatus according to an embodiment of the present invention.

The skin care apparatus 1 may include at least some or all of the vibration module 30, the temperature control module 40, the ion module 42, the far-infrared ray module 44, the light emitting module 46, the communication module 50, the power module 60, a measurement module 70, and a processor 90.

The processor 90 may control at least one of the vibration module 30, the temperature control module 40, the ion module 42, the far-infrared ray module 44, the light emitting module 46, the communication module 50, the power module 60, and the measurement module 70.

The stimulation providing unit may include the vibration module 30, the temperature control module 40, the ion module 42, the far-infrared ray module 44, the light emitting module 46, and the like. However, it is merely illustrative, and the skin care apparatus 1 according to the embodiment of the present invention may further include various modules capable of providing stimulation to the skin.

The temperature control module 40, the ion module 42, the far-infrared module 44, the light emitting module 46, and the like may be disposed between the outer body 10 and the inner body 20. Alternatively, at least some of the temperature control module 40, the ion module 42, and the far-infrared ray module 44 may be disposed on the inner surface 21 of the inner body 20.

The temperature control module 40 may include an element that generates a heating or cooling effect using the power supplied from the power module 60.

For example, the skin care apparatus 1 may include a plurality of temperature control modules 40. The plurality of temperature control modules 40 may be disposed in the first area A1, and when the temperature control module 40 disposed in the first area A1 generates heat, there is an effect capable of promoting the absorption of the ingredients contained in the sheet 2 in contact with the first area A1 into the skin.

However, it is merely illustrative, and the temperature control module 40 may have various positions as described above and generate a cooling effect as well as a heating effect. The ion module 42 may conduct electricity with the skin to supply at least one of positive and negative ions to the skin. The skin care apparatus 1 may remove skin impurities by supplying positive ions to the skin and circulate blood by supplying negative ions to the skin.

The ion module 42 may supply positive ions or negative ions through an alternating current or supply positive ions or negative ions through a galvanic current. In particular, when the ion module 42 supplies positive ions or negative ions to the skin through the galvanic current, there is an advantage of supplying positive ions or negative ions to the skin while minimizing skin irritation.

The processor 90 may control so that the ion module 42 generates only positive ions in a positive ion mode, the ion module 42 generates only negative ions in a negative ion mode, and the ion module 42 generates the positive ions and negative ions alternately in a complex mode.

In addition, the ion module 42 may further generate galvanic ions.

The far-infrared module 44 may emit far-infrared light in a wavelength band having a high transmission performance among infrared rays, and through this, it is possible to transmit light energy of the far-infrared light to the inside of the skin. The far-infrared light may penetrate the skin even though it is not in direct contact with the skin, and thus there is an advantage that the light energy may be effectively transmitted.

The light emitting module 46 may include an LED. The light emitting module 46 may differently adjust a color emitted from the LED according to a care mode. For example, the light emitting module 46 may emit red light in a first care mode, emit green light in a second care mode, emit blue light in a third care mode, and sequentially emit red light, green light, and blue light in a fourth care mode. As another example, the light emitting module 46 may emit red light, green light, and blue light individually or in combination.

Meanwhile, in the present invention, the temperature control module 40 and the light emitting module 46 have been described as separate configurations, but it is only described separately to distinguish functions, and thus it is reasonable that the embodiment is not limited thereto.

The stimulation providing unit may operate by receiving a control signal from a terminal 3 or a server 5 to be described later.

The measurement module 70 may include an impedance sensor for measuring moisture and oil of the skin of the user. The measurement module 70 may measure moisture, oil, and the like of the skin of the user through a measured value of the impedance sensor.

In addition, the measurement module 70 may further include an ultrasonic transducer and an ultrasonic sensor. The ultrasonic transducer may emit an ultrasonic beam in a form of a pulse and the ultrasonic sensor may sense that a beam emitted by the ultrasonic transducer is reflected at a boundary of the skin tissue to measure the skin thickness. It will be described in more detail in FIG. 17.

In addition, the measurement module 70 may further include a current sensor that senses an amount of current flowing through one region of the inner surface 21. The current sensor monitors the amount of current to determine whether the user is mounted with the skin care apparatus 1. For example, the current sensor may determine that the user has mounted or removed the skin care apparatus 1 when the amount of current is changed to the set value or more.

The processor 90 may set and control the vibration intensity and the number of vibrations of each of the plurality of vibration modules 30. In addition, the processor 90 may set and control the heat generation temperature of each of the temperature control modules 40.

The processor 90 may transmit and receive a signal to and from the terminal 3 or the server 5 to be described later through the communication module 50.

The processor 90 may supply power to the vibration module 30, the temperature control module 40, the communication module 50, and the processor 90 through the power module 60.

Meanwhile, the skin care apparatus 1 may further include other components in addition to the components described in FIG. 11. Hereinafter, various components that the skin care apparatus 1 may have will be described.

For example, the skin care apparatus 1 may further include a piezoelectric sensor 410, which will be described in detail with reference to FIG. 12. The piezoelectric sensor 410 may be connected to the circuit board 99.

FIG. 12 is a view illustrating a piezoelectric sensor that may be provided in the skin care apparatus according to the embodiment of the present invention.

The piezoelectric sensor 410 may include a flexible substrate 410a, a piezoelectric thin film layer 410b on the flexible substrate 410a, an electrode 410c on the piezoelectric thin film layer 410b, and a protective layer 410d on the electrode 410c.

The piezoelectric thin film layer 410b may include an inorganic material having a perovskite structure. For example, the piezoelectric thin film layer 410b may include a material such as BaTiO3, LiNbO3, LiTaO3, KNbO3, and the like, but the embodiment is not limited thereto.

A skin care apparatus according to the related art has a problem that treatment or care is uniformly performed with a function set in the product itself rather than considering skin elasticity of the user to be treated.

In particular, in the related art, it is difficult to check in real time how much skin elasticity is improved during skin care.

Accordingly, the skin care apparatus 1 according to the present invention includes the piezoelectric sensor 410 having the piezoelectric thin film layer 410b disposed on the flexible substrate 410a, and accordingly, there is a special technical effect that it is possible to measure skin elasticity in real time during skin care.

For example, there is a special technical effect that it is possible to measure skin elasticity in real time during skin care by detecting minute changes in skin elasticity through the piezoelectric thin film layer 410b and the electrode 410c.

In order to increase the sensitivity of the skin elasticity change, a signal amplifying unit, a signal filtering unit, and the like may be further included.

FIG. 13 is a view illustrating an inner surface of a main body according to another embodiment of the present invention.

According to the embodiment, an adhesive layer 21a may be formed on an edge of inner surface 21, and in this case, it is possible to increase the effect of skin care by increasing the adhesion between the skin care apparatus 1 and the facial skin.

For example, the adhesion to the skin is increased by arranging the adhesive layer 21a made of an acrylic monomer with high adhesion and biocompatibility on the edge of the inner surface 21, and accordingly, the adhesion between the face mask and the facial skin is increased, thereby increasing the effecting of skin care.

The sheet 2 is positioned in the sheet accommodation part 110, but the sheet 2 including a cosmetic formulation is more closely adhered to the face by disposing the adhesive layer 21a on the edge of the inner surface 21, and accordingly, the effect of skin care is increased by active ingredients, and the adhesion between the face mask and the facial skin is increased, thereby increasing the effect of skin care.

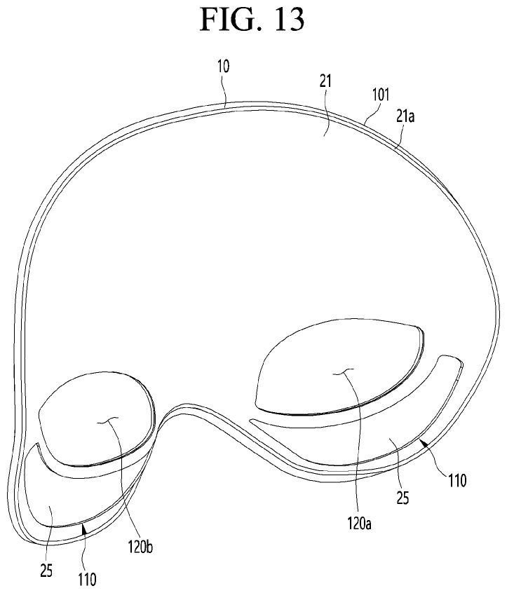

In addition, the skin care apparatus 1 may further include a contact time sensing device 420, which will be described in detail with reference to FIG. 14. The contact time sensing device 420 may be connected to the circuit board 99.

FIG. 14 is a view illustrating the contact time sensing device that may be provided in the skin care apparatus according to the embodiment of the present invention.

The face mask may include the inner surface 21, in particular, the contact time sensing device 420 disposed on the sheet accommodation part 110.

The contact time sensing device 420 may include a monitor module 421, a plurality of electrical contacts 422, and a plurality of ports 423. The plurality of electrical contacts 422 may be connected to the monitor module 421 by a power line EL.

According to the embodiment, face care may be performed using the sheet 3 containing the cosmetic formulation.

At this time, the cosmetic formulation contains moisture, and the moisture decreases as it is used.

Accordingly, there is a special technical effect that can check in real time the degree of use of the mask sheet 2 and the progress of the skin care time by measuring a moisture content of the cosmetic formulation contained in the sheet 2.

For example, the contact time sensing device 420 may maintain a voltage on a first electrical contact point of the port 423 and detect a short-circuit between the first electrical contact point and a second electrical contact point of the port, and when the monitor module 421 detects the short-circuit, the contact time sensing device 420 may determine that the cosmetic formulation of the sheet 2 remains sufficiently, and when the short-circuit is not detected, the contact time sensing device 420 may determine in real time that the cosmetic formulation is insufficient.

Through this, there is a special technical effect that it is possible to check the degree of use of the sheet 2, the progress of skin care time, and the like in real time by accurately measuring the moisture content of the cosmetic formulation contained in the sheet 2 through the contact time sensing device 420.

Meanwhile, according to the related art, a light emitting device and a vibration device for skin care are applied in a form of individual devices.

However, when the light emitting device and the vibration device are disposed as separate devices, there is a technical contradiction that uniform care may not be implemented over the entire face because the light emitting device may not be disposed in an area where the vibration device is disposed.

In order to overcome such a technical contradiction, the skin care apparatus 1 may include an optical vibration composite device 430 disposed on the inner surface 21. The optical vibration composite device 430 may be connected to the circuit board 99.

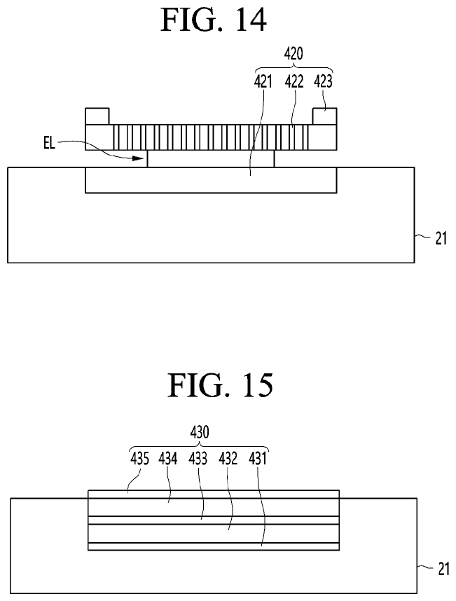

FIG. 15 is a view illustrating an optical vibration composite device that may be provided in the skin care apparatus according to the embodiment of the present invention.

For example, the optical vibration composite device 430 may include a first electrode layer 431 disposed on the inner surface 21, a flexible light emitting layer 432 disposed on the first electrode layer 431, a second electrode layer 433 disposed on the flexible light emitting layer 432, a flexible ultrasonic layer 434 disposed on the second electrode layer 433, and a third electrode layer 435 disposed on the flexible ultrasonic layer 434.

The first electrode layer 431 and the third electrode layer 435 may be an anode, and the second electrode layer 433 may be a common cathode.

DC power may be applied to the flexible light emitting layer 432, and AC power may be applied to the flexible ultrasonic layer 434.

The flexible light emitting layer 432 may be an OLED, and the flexible ultrasonic layer 434 may be a flexible piezo coating layer, but the embodiment is not limited thereto.

There is a technical effect that it is possible to simultaneously implement light and ultrasonic waves for facial care by providing the optical vibration composite device 430 including the flexible light emitting layer 432 and the flexible ultrasonic layer 434.

In particular, as light and ultrasonic waves may be simultaneously implemented by one composite device 430, the optical vibration composite device 430 may be densely disposed over the entire face, and accordingly, there is a special technical effect that it is possible to implement very uniform care over the entire face.

Meanwhile, according to the related art, when an LED is used in a skin care apparatus, a single wavelength LED is used or an LED capable of emitting individual wavelengths is used even though an LED of a plurality of various wavelengths are used.

Accordingly, the number of LEDs that may be mounted is limited in the conventional LED mask, and there was a limitation that an emission wavelength may not be used in various ways according to needs of a user.

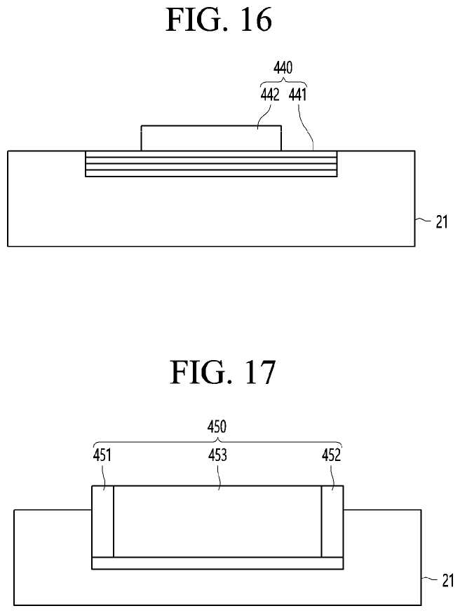

FIG. 16 is a view illustrating a tunable light emitting module that may be provided in the skin care apparatus according to the embodiment of the present invention.

The skin care apparatus 1 may include a wavelength tunable light emitting module 440 as shown in FIG. 16. The wavelength tunable light emitting module 440 may include a piezoelectric substrate 441 disposed on a circuit board 315 and a wavelength tunable light emitting device 442 disposed on the piezoelectric substrate 441.

For example, the skin elasticity of the user may be measured by including the piezoelectric sensor 410 including the piezoelectric thin film layer 410b disposed on the flexible substrate 410a described above.

There is a special technical effect that enables skin care by a corresponding LED wavelength after measuring the elasticity of the skin of the user through the piezoelectric sensor 410 before skin care and then selecting an appropriate LED wavelength according to the user.

Alternatively, it may be varied to an appropriate wavelength according to a care purpose of the user. For example, in the case of atopic dermatitis care, a wavelength of about 617 nm may be effective, and in the case of acne care, a wavelength of 630 nm may be effective.

A wavelength of the wavelength tunable light emitting device 442 may be varied according to a change in the phenomenon of the piezoelectric substrate 441.

For example, a voltage is applied to the piezoelectric substrate 441 to generate mechanical stress, and the mechanical stress may change a wavelength of output light of the wavelength tunable light emitting device 442, and the piezoelectric substrate 441 may use piezoelectric ceramic, crystal, or the like, but the embodiment is not limited thereto.

For example, the piezoelectric substrate 441 includes a first piezoelectric layer (not shown) and a second piezoelectric layer (not shown) disposed on the first piezoelectric layer, and when the second piezoelectric layer expands in the horizontal direction in a state in which the first piezoelectric layer is not deformed, the piezoelectric substrate 441 may be bent in a convex shape toward thereabove.

Bending occurs also in the wavelength tunable light emitting device 442 disposed thereon by bending the piezoelectric substrate 441 as described above.

In this case, an active layer (not shown) is formed on the wavelength tunable light emitting device 442, and expansion and deformation occurs in the active layer of the light emitting device 442 as well. The light emitting device 442 generates spontaneous polarization according to a stacked structure of atoms composing a compound semiconductor and piezoelectric polarization due to lattice mismatch between a well layer and a barrier layer when the active layer is grown.

When expansion and deformation occur in the active layer due to the convex bending, the polarization effect of the active layer decreases and carriers (electrons and holes) capable of emission recombination increase, and accordingly, there is a characteristic that the emission wavelength shifts to a shorter wavelength as the emission energy increases.

On the other hand, bending occurs so that the piezoelectric substrate 441 has a convex shape toward therebelow, the active layer of the light emitting device 442 is contracted and deformed in a plane direction, and the polarization phenomenon inside the active layer is intensified and carriers (electrons and holes) capable of emission recombination are reduced, and accordingly, the emission wavelength may shift in a long wavelength direction as the emission energy decreases.

There is a special technical effect that it is possible to perform skin care by a corresponding LED wavelength after measuring the skin elasticity of the user through the piezoelectric sensor 410 before skin care and selecting an appropriate LED wavelength according to the user.

Meanwhile, the skin care apparatus 1 may further include a skin thickness measurement device 450.

FIG. 17 is a view illustrating a skin thickness measurement device that may be provided in the skin care apparatus according to the embodiment of the present invention.

The skin thickness measurement device 450 may include one or more ultrasonic transducers 453 disposed on the inner body 20 and one or more RF electrodes 451 and 452 disposed on both sides of the ultrasonic transducer 453.

A thickness of an adipose tissue layer of the skin of the user may be accurately measured before skin care, and through this, the wavelength tunable light emitting module 440 is organically combined to control an optimal LED wavelength suitable for the user to perform skin care, and accordingly, there is a technical effect that may maximize the effect of skin care.

For example, a through-hole (not shown) may be provided in a part of the inner body 20 or the sheet 2 so that the skin thickness measurement device 450 may be in contact with the skin of the user, but the embodiment is not limited thereto.

For example, the ultrasonic transducer 453 of the skin thickness measurement device 450 and one or more RF electrodes may be coupled to a surface of a skin layer of the user with a predetermined level of force.

For example, electrical impedance of an adipose tissue segment of the skin of the user disposed between a first electrode 451 and a second electrode 452 may be measured by applying a preset voltage between the first electrode 451 and the second electrode 452 placed on the surface of the skin of the user.

For example, a current applied to the first electrode 451 forms a current path along each thickness of the skin layer of the user to flow in the skin tissue, and it is possible to determine the conductivity or impedance of the adipose tissue segment of the user by measuring the total current at an electrode-skin surface coupling point, and through this, a skin thickness of an adipose tissue of the user may be measured.

Accordingly, the thickness of the adipose tissue layer of the skin of the user may be accurately measured before skin care, and through this, the wavelength tunable light emitting module 440 is organically combined to control the optimal LED wavelength suitable for the user to perform skin care, and accordingly, there is a technical effect that may maximize the effect of skin care.

Meanwhile, the skin care apparatus 1 may further include a linear ultrasound module 460.

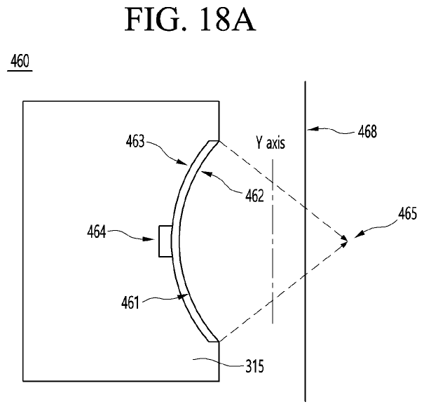

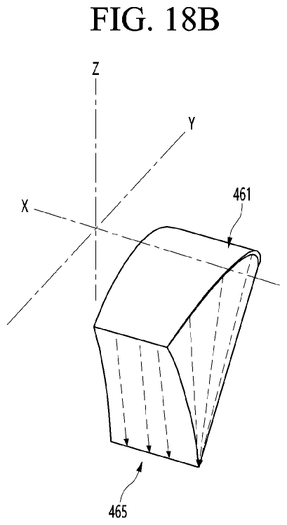

FIGS. 18A to 18B are views illustrating a linear ultrasound module that may be provided in the skin care apparatus according to the embodiment of the present invention.

For example, the linear ultrasound module 460 may include a cylindrical conversion element 461 disposed on the inner body 20 and an imaging element 464 disposed on the cylindrical conversion element 461.

For example, as described above, the thickness of the adipose tissue layer of the skin of the user may be accurately measured before skin care.

Thereafter, since it is possible to generate linear ultrasonic waves using the linear ultrasound module 460, there is a special technical effect that may exert an intensive and very optimal care effect in consideration of the skin thickness of the user.

For example, referring to FIG. 18A, the linear ultrasound module 460 may include the cylindrical conversion element 461 and the imaging element 464, and the cylindrical conversion element 461 may have a concave surface 462 and a convex surface 463.

The cylindrical conversion element 461 may be cut opened to show the release of energy from the concave surface 462 to a treatment area 465 in a user skin surface 468.

Referring to FIG. 18B, the ultrasound generated by the linear ultrasound module 460 may include a spherical shaped bowl shape having at least one of geometrically concentrated concave surface having a predetermined width (X-axis) and height (Y-axis) in a single point treatment area 465 at a focal depth (Z-axis) under the skin surface 468.

The linear ultrasonic waves may be generated using the linear ultrasound module 460, and after accurately measuring the skin thickness of the user according to the tenth embodiment, the linear ultrasound module 460 is used in consideration of the skin thickness of the user and a depth of treatment or care, and accordingly, there is a special technical effect that may exert an intensive and very optimal care effect.

Meanwhile, the skin care apparatus 1 may further include a composite skin thickness measurement device 470.

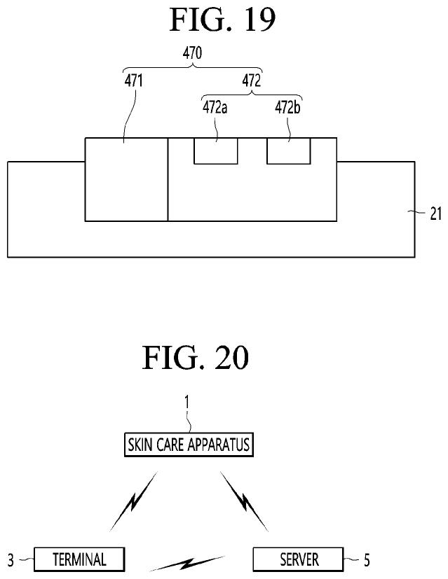

FIG. 19 is a view illustrating a composite skin thickness measurement device that may be provided in the skin care apparatus according to the embodiment of the present invention.

The composite skin thickness measurement device 470 may include an ultrasound module 471 and a light detection module 472.

The ultrasound module 471 may adopt the skin thickness measurement device 450 described above.

In addition, the light detection module 472 may include a light emitting part 472a and a light sensor 472b. In addition, the light emitting part 472a may be an LED, and the light sensor 472b may be a photodiode.

The skin thickness may be measured by detecting a time of light reflected from the skin of the user of light emitted from the light emitting part 472a.

The skin thickness of the user may be accurately measured by the ultrasound module 471 and the light detection module 472, and through this, the wavelength tunable light emitting module 440 is organically combined to control the optimal LED wavelength suitable for the corresponding user and perform skin care, and accordingly, there is a technical effect that may maximize the effect of skin care.

In addition, after accurately measuring the skin thickness of the user using the linear ultrasound module 460 and using the linear ultrasound module 460 in consideration of the skin thickness of the user and treatment or care depth, and accordingly, there is a special technical effect that may exert an intensive and very optimal care effect.

FIG. 20 is a control block diagram of a skin care apparatus system including the skin care apparatus according to the embodiment of the present invention.

The skin care apparatus system may include the skin care apparatus 1, the terminal 3, and the server 5. Each of the skin care apparatus 1, the terminal 3, and the server 5 may transmit and receive a signal to and from each other. The skin care apparatus 1 may transmit and receive a signal to and from the terminal 3 or the server 5 through the communication module 50.

The terminal 3 may include a smart phone, a personal computer (PC), a notebook, and the like.

Meanwhile, although only one server 5 is shown in FIG. 20, the server 5 may be implemented in plural according to a function to be described later.

According to the embodiment of the present invention, the server 5 may acquire environmental information.

The environmental information may refer to information related to the atmosphere and may include various information that may affect the human skin. For example, the environmental information may include at least one of an ultraviolet index, a temperature, a humidity, an air pollution level, an air pollution position, and the like.

The server 5 may acquire a method of operating the skin care apparatus 1 based on the environmental information. The operation method may refer to operation intensity or an operation time of at least one of the vibration module 30 and the temperature control module 40 provided in the skin care apparatus 1.

For example, the server 5 may acquire an operation method of setting the heat generating intensity of the temperature control module 40 to “weak” when the temperature is 30° C. or more, the heat generating intensity of the temperature control module 40 to “medium” when the temperature is 20° C. or more and less than 30° C., and the heat generating intensity of the temperature control module 40 to “strong” when the temperature is less than 20° C.

In addition, the skin care apparatus 1 may have a plurality of care modes, and each of the plurality of care modes may set an operation strength or operation time of the vibration module 30 and an operation strength or operation time of the temperature control module 40 to be different. The server 5 may acquire the operation method of the skin care apparatus 1 by determining any one of the plurality of care modes based on the environmental information.

The server 5 may transmit the operation method of the skin care apparatus 1 acquired based on the environmental information to the skin care apparatus 1.

Meanwhile, it has been described that a subject that acquires the operation method of the skin care apparatus 1 according to the environmental information is the server 5, but it is merely illustrative. That is, after the terminal 3 acquires the environmental information, the terminal 3 may acquire the operation method of the skin care apparatus 1 based on the environmental information to transmit the operation method to the skin care apparatus 1, or the terminal 3 may acquire the operation method of the skin care apparatus 1 based on the environmental information after the skin care apparatus 1 acquires the environmental information.

According to another embodiment of the present invention, the terminal 3 may acquire the environmental information and the user information. Since the environmental information is the same as described above, duplicated descriptions will be omitted.

The user information refers to information related to the skin and skin care of the user and may include skin information of the user, a user's usage history of the skin care apparatus, and the like. For example, the skin information may include oil content, moisture content, skin elasticity, wrinkle information, etc. of the skin of the user's face, and the usage history of the skin care apparatus may include the number of times the skin care apparatus 1 has been used, cycles, a date of recent use, preferred strength and number of times of the vibration module 30, and preferred strength and time of the temperature control module 40.

The terminal 3 may acquire the operation method of the skin care apparatus 1 by considering the environmental information and the user information together and transmit the acquired operation method to the skin care apparatus 1. The operation method has been described above, and thus a duplicated description will be omitted.

Meanwhile, it has been described that the subject that acquires the operation method of the skin care apparatus 1 according to the environmental information and the user information is the terminal 3, but it is merely illustrative. That is, after the server 5 acquires the environmental information and the user information, the server 5 may acquire the operation method of the skin care apparatus 1 to transmit the operation method to the skin care apparatus 1, or the server 5 may acquire the operation method of the skin care apparatus 1 after the skin care apparatus 1 acquires the environmental information and the user information.

According to still another embodiment of the present invention, the skin care apparatus 1 may receive an operation command from the terminal 3.

The terminal 3 may include an input unit (not shown) for receiving an operation input from the user, and the terminal 3 may transmit the operation command to the skin care apparatus 1 so that the skin care apparatus 1 operates according to the operation input received from the input unit (not shown).

As an example, the terminal 3 may display a plurality of care modes and receive an input for selecting any one of the plurality of care modes through the input unit (not shown).

The above-described descriptions are merely illustrative of the technological spirit of the present invention, and various modifications and variations may be made by those having ordinary skill in the art to which the present invention pertains without departing from the essential characteristics of the present invention.

Therefore, the embodiments disclosed in the present invention are not intended to limit the technological spirit of the present invention, but the embodiments are intended to describe, and the spirit and scope of the present invention is not limited by such embodiments.

The protection scope of the present invention should be construed by the following claims, and all technological spirits within the equivalent scope thereof should be construed as being included in the scope of right of the present invention.

1. A skin care apparatus comprising:
a main body; and
a mounting body for mounting the main body on a user's face,
wherein the main body is formed with a sheet accommodation part in which a sheet is accommodated,
the main body includes at least one first area with which the sheet accommodated in the sheet accommodation part is in contact, and a second area between an edge of the main body and the first area, and
a thickness of the first area is smaller than a thickness of the second area.

2. The skin care apparatus of claim 1, wherein the thickness of the first area is an average thickness of the first area, and
the thickness of the second area is an average thickness of the second area.

3. The skin care apparatus of claim 1, wherein the main body is formed with an opening corresponding to the user's eyes, and
at least a part of the first area overlaps the opening in a vertical direction.

4. The skin care apparatus of claim 1, wherein the first area is provided to the main body in pairs,
the pair of first areas are spaced apart from each other in left and right directions, and the main body includes:
a left area including the first area positioned on the left of the pair of first areas;
a right area including the first area positioned on the right of the pair of first areas; and
a central area between the left area and the right area,
wherein an average thickness of the central area is greater than each of an average thickness of the left area and an average thickness of the right area.

5. The skin care apparatus of claim 1, wherein the main body further includes:
an outer body;
an inner body disposed on a rear surface of the outer body and having an inner surface facing the user's face; and
a vibration module disposed between the outer body and the inner body.

6. The skin care apparatus of claim 5, wherein a thickness of the inner body is smaller than a thickness of the outer body.

7. The skin care apparatus of claim 1, wherein the main body further includes:
an outer body;
an inner body disposed on a rear surface of the outer body and having an inner surface facing the user's face; and
a heat generating module disposed between the outer body and the inner body.

8. The skin care apparatus of claim 7, wherein a thickness of the inner body is smaller than a thickness of the outer body.

9. The skin care apparatus of claim 7, wherein the heat generating module overlaps the first area in a horizontal direction.

10. The skin care apparatus of claim 1, wherein the sheet accommodation part further includes a rib for guiding the mounting of the sheet.

11. The skin care apparatus of claim 10, wherein a thickness of the rib is smaller than a thickness of the sheet.

12. The skin care apparatus of claim 1, wherein the sheet accommodation part is formed with a recessed portion for accommodating the sheet.

13. The skin care apparatus of claim 12, wherein a depth of the recessed portion is smaller than the thickness of the sheet.

14. The skin care apparatus of claim 1, wherein the sheet is detached/attached from/to the sheet accommodation part.

15. The skin care apparatus of claim 1, wherein the main body is formed of silicone, thermo plastic elastomer (TPE), or urethane.

16. A skin care apparatus system comprising:
a terminal; and
a skin care apparatus including a communication module for transmitting and receiving a signal to and from the terminal,
wherein the skin care apparatus includes:
a main body; and
a mounting body for mounting the main body on a user's face, and
the main body is formed with a sheet accommodation part in which a sheet is accommodated,
the main body includes at least one first area with which the sheet accommodated in the sheet accommodation part is in contact, and a second area between an edge of the main body and the first area, and
a thickness of the first area is smaller than a thickness of the second area.

17. The skin care apparatus system of claim 16, wherein the skin care apparatus further includes:
a vibration module mounted on the main body; and
a processor for setting an operation method of the vibration module based on a control command received from the terminal.

18. The skin care apparatus system of claim 17, wherein the processor sets vibration intensity of the vibration module disposed at a position corresponding to the first area to be different from vibration intensity of the vibration module disposed at a position corresponding to the second area.

19. The skin care apparatus system of claim 16, wherein the skin care apparatus further includes:
a heat generating module mounted on the main body; and
a processor for setting an operation method of the heat generating module based on a control command received from the terminal.

20. The skin care apparatus system of claim 16, wherein the skin care apparatus further includes:
a processor for setting an operation method based on at least one piece of environmental information and user information received from the terminal.
